(19) United States
(12) Patent Application Publication
(10) Pub. No.: US 2017/0000830 A1
(43) Pub. Date: Jan. 5, 2017

(54) Compositions for Relieving the Symptoms of Gluten Sensitivity and Methods of Use Thereof

(71) Applicant: Rakesh Saini, Farmington Hills, MI (US)

(72) Inventor: Rakesh Saini, Whitehouse Station, NJ (US)

(21) Appl. No.: 15/200,498

(22) Filed: Jul. 1, 2016

Related U.S. Application Data
(60) Provisional application No. 62/187,352, filed on Jul. 1, 2015.

(51) Int. Cl. A61K 35/747; A61K 38/46; A61K 38/54; A61K 36/9068; A61K 36/9066; A61K 38/48; A61K 38/47

(52) U.S. Cl. CPC A61K 35/747; A61K 38/48; A61K 38/4873; C12Y 304/22002; C12Y 304/22032; A61K 38/482; C12Y 304/21063; A61K 38/465; A61K 38/47; C12Y 302/01108; C12Y 302/01022; C12Y 302/01006; C12Y 302/01004; C12Y 302/01008; C12Y 302/01003; C12Y 302/01001; A61K 36/9068; A61K 36/9066; C12Y 302/01026; A61K 38/54

(57) ABSTRACT

The disclosure relates to a composition that may be used to prevent the symptoms of non-celiac gluten sensitivity and may include ingredients that degrade or inactivate gluten and other components that may cause these symptoms.

The presence of wheat or other grains, such as rye and barley, in the diet may trigger adverse reactions in certain individuals. For example, celiac disease is an immune condition triggered by the presence of the protein gluten, and possibly other molecules, found in wheat and other grains. Celiac disease occurs largely in individuals with a well-defined genetic profile and results in digestive system-related symptoms (e.g. constipation, diarrhea) as well as the production of antibodies to gluten and permanent damage to the intestine. Another group of individuals experience non-celiac gluten sensitivity which presents with similar digestive symptoms but appears to not have a well-defined genetic component and does not result in the production of antibodies or intestinal damage. The term gluten is understood generally to describe a family of proteins that are implicated in causing the symptoms of the celiac disease, non-celiac gluten sensitivity and similar disorders.

The avoidance of gluten in an individual's diet will relieve the symptoms of non-celiac gluten sensitivity and other related disorders. However, this requires constant vigilance on the part of the individual which can be quite onerous. Consequently, there is a need for an effective composition that would prevent or relieve the symptoms of non-celiac gluten sensitivity and other gluten-related disorders when taken by affected individuals before or during the ingestion of food.

### SUMMARY

According to one example, the disclosure relates to a composition that includes at least three proteases, at least one carbohydrase and at least one probiotic wherein said composition relieves symptoms of gluten sensitivity.

According to one example, the disclosure relates to a composition for preventing the effects of gluten in a food product, wherein said composition includes at least three proteases selected from Protease 3.0, Protease 4.5, Protease 6.0, Papain, Bromelain, Protease Aspergillus oryzae, Protease Aspergillus melleus, Proteases I-V, and DPP IV and combinations, a least one carbohydrase selected from lactase, α-galactosidase, β-glucanase, xylanase, glucoamylase, amylase, hemicellulase, invertase and pectinase, at least one probiotic selected from Lactococcus lactis and Lactococcus cremoris, ginger, turmeric, lipase, and phytase. The composition reduces the concentration of gluten to less than 20 ppm.

According to one example, the disclosure relates to a method of reducing non-celiac gluten sensitivity including formulating a composition, where the composition includes at least three proteases selected from Protease 3.0, Protease 4.5, Protease 6.0, Papain, Bromelain, Protease Aspergillus oryzae, Protease Aspergillus melleus, Proteases I-V, and DPP IV, a least one carbohydrase selected from the group consisting of lactase, α-galactosidase, β-glucanase, xylanase, glucoamylase, amylase, hemicellulase, invertase and pectinase, a least one probiotic from Lactococcus lactis and Lactococcus cremoris, ginger, turmeric, lipase, and phytase, ingesting the composition, such that the composition relieves the symptoms of non-celiac gluten sensitivity.

### BRIEF DESCRIPTION OF DRAWINGS

FIG. 1 is a bar graph demonstrating the effects of a composition according to the disclosure on the degradation of gluten in bread and pasta samples.

### DETAILED DESCRIPTION

It is to be understood that the disclosed examples and specific details disclosed herein are not to be interpreted as limiting, but merely as a representative basis for any aspect of the disclosure and/or as a representative basis for teaching one skilled in the art to variously employ the compositions and methods of the disclosure. Furthermore, the terminology used herein is used only for the purpose of describing particular examples according to the disclosure and is not intended to be limiting in any way

Except in the Examples and where otherwise expressly indicated, all numerical quantities in this description indicating amounts of material or conditions of reaction and use are to be understood as modified by the word “about” in describing the broadest scope of the disclosure.

The present disclosure relates generally compositions and methods of using the compositions that relieve, reduce and prevent the symptoms of an gluten sensitive-individual when that individual ingests a gluten-containing product.

In one example, a composition according to the disclosure relieves or reduces a gluten-sensitive individual's symptoms of discomfort when the individual ingests one or more product(s) containing gluten or gluten-related molecules. In a further example, a composition according to the disclosure prevents an individual's symptoms when the individual ingests one or more product(s) containing gluten or gluten-related molecules.

In one example, a composition according to the disclosure relieves, reduces and prevents the symptoms of non-celiac gluten sensitivity when a gluten-sensitive individual ingests food or beverage products containing gluten or gluten-related molecules. For example, the gluten and gluten-related molecules may be a component of a gluten-containing flour that is used in a food product, including, without limitation, wheat, rye and barley flour and including products that use a combination of these ingredients. The composition is effective when an individual ingests a product containing gluten including, without limitation, bread products, pasta, cereal, cookies, cakes and other baked products, processed food products containing gluten and beverages such as beer.

In a further example, a composition according to the disclosure, modifies or alters a food or beverage product such that the product does not cause the symptoms of non-celiac sensitivity or such that the product causes reduced symptoms or effects.

In a further example, a composition according to the disclosure provides relief from at least one symptom of abdominal pain, cramping, bloating/distention, diarrhea, constipation and fatigue that result from non-celiac gluten sensitivity.

In a further example, a composition according to the disclosure inactivates, modifies and or otherwise alters molecules found in a found or beverage product that directly cause or are implicated in the symptoms of non-celiac gluten sensitivity. The inactivation, modification or alteration of such molecules results in reduced non-celiac gluten sensitivity or relief from non-celiac gluten sensitivity.

In one example, a composition according to the disclosure includes proteases and peptidases that inactivate, degrade and partially inactivate proteins, including the protein components of glycoproteins and lipoproteins and other molecules where proteins and non-protein components are covalently bonded or otherwise bonded or co-assembled.

In one example, a composition according to the disclosure includes at least three proteases where the proteases have least some non-overlapping specificity for cleavage of amino acid chains. In another example, the composition includes at least three proteases that have at least some non-overlapping specific activities under a given set of conditions.

In preferred examples, the compositions according to the disclosure include multiple proteases that degrade gluten and related proteins that cause non-celiac sensitivity and other gluten-induced disorders. In a further example, a composition according to the disclosure contains at least three proteases that proteolyze, degrade, partially degrade or otherwise inactivate gluten and gluten-related molecules. In further examples, the composition according to the disclosure includes at least three or peptidases, or at least four proteases or peptidases, at least five proteases or peptidases, or at least six proteases or peptidases, or at least seven proteases or peptidases or at least many eight proteases or peptidases that degrade gluten and other proteins. The proteases may have similar or overlapping specificities for proteins or may have unique specificities. In a preferred example, the proteases and peptidases are selected from the group consisting of Protease 3.0, Protease 4.5, Protease 6.0, Papain, Bromelain, Protease Aspergillus oryzae, Protease Aspergillus melleus, Proteases I-V, and DPP IV.

In some examples, proteases are present in the composition from about 0.0001% to about 50% by weight of the composition. In further examples proteases and peptidases are present in the composition from about 0.0001% to about 10% by weight of the composition. In some examples, proteases and peptidases are present in the composition from about 0.0001% to about 5% by weight of the composition. In some examples proteases and peptidases are present in the composition from about 0.0001% to about 50% by weight of the composition. In some examples proteases and peptidases, proteases are present in the composition from 0.001% to 2% by weight of the composition.

The compositions according to the disclosure may include at least one carbohydrase, or at least two carbohydrases, or at least three carbohydrases, at least four carbohydrases, or at least five carbohydrases, or at least six carbohydrases, or at least seven carbohydrases or at least eight carbohydrases, or at least nine carbohydrases or at least ten carbohydrases or at least eleven carbohydrases. The carbohydrases may have similar or overlapping specificities for carbohydrate or may have unique specificities. In a preferred example, the carbohydrases are selected from the group consisting of lactase, α-galactosidase, β-glucanase, xylanase, glucoamylase, amylase, hemicellulase, invertase and pectinase.

In one embodiment, carbohydrases are present in the composition from about 0.0001% to about 50% by weight of the composition. In further embodiments carbohydrases are present in the composition from about 0.0001% to about 10% by weight of the composition. In one example, carbohydrases are present in the composition from about 0.0001% to about 5% by weight of the composition. In one example, proteases are present in the composition from about 0.001% to about 2% by weight of the composition.

In one example, a composition according to the disclosure includes both proteases and carbohydrases. In a preferred example, a composition according to the disclosure includes at least three proteases and at least one carbohydrase. In a preferred example, a composition according to the disclosure includes at least three proteases and at least two carbohydrase. In a preferred example, a composition according to the disclosure includes at least three proteases and at least three carbohydrases.

In a preferred example, a composition according to the disclosure reduces the concentration of gluten found in food products such that the gluten concentration is reduced to below about 20 ppm, or below about 10 ppm or below about 5 ppm. In other embodiments, the gluten concentration may be in the range of about 0.1 ppm to about 20 ppm, 0 about 0.1 ppm to about 10 ppm, or about 0.1 ppm to about 5.0 ppm. The Food and Drug Administration requires that a product be less than 20 ppm to be labeled gluten-free.

The compositions according to the disclosure may have at least one lipase. The composition according to the disclosure at least one or more probiotics. In a preferred example, the probiotics are selected from the group consisting of Lactococcus lactis and Lactococcus cremoris. The compositions according to the disclosure may include other ingredients such as ginger or turmeric. The compositions according to the disclosure may include phytase.

In one example according to the disclosure, a composition may be used in a method to treat the symptoms of non-celiac gluten sensitivity. In a preferred example, the composition may be ingested by an individual prior to the consumption of a food or beverage product. In a further example, the composition is ingested simultaneously with the ingestion of a food or beverage product.

The composition according to the disclosure may be taken as a dietary supplement. The dietary supplement may be in solid form, including, without limitation, tablets, capsules, powders, pills, candy and bars. The composition may include flavorants, excipients and preservatives.

The composition according to the disclosure may be incorporated into other supplements or other products. The compositions according to the disclosure may be taken simultaneously with other supplements, with food or with beverages.

### Example 1

An exemplary composition according to the disclosure within the scope of the disclosure was tested for its ability to degrade gluten present in two common food samples, pasta and whole wheat bread. The composition used in these experiments is outlined in Table 1. The experimental conditions were chosen to mimic the conditions within the stomach during digestion of food.

TABLE 1

| Component | Activity | |
|---|---|---|
| Protease 3.0 | 910 | SAPU/g |
| Protease 4.5 | 425,000 | HUT/g |
| Protease 6.0 | 425,000 | HUT/g |
| Papain | 50,000 | PU/mg |
| | 27,000 | PU/mg 2000 |
| Bromelain | GDU/g | |
| Protease Aspergillus oryzae | 30,000 | HUT/g |
| Protease Aspergillus oryzae | 500 | DPP-IV |
| Protease Aspergillus melleus | 6.5 | AP |
| Lipase | 20,000 | LU/g |
| Lactase | 100,000 | ALU/g |
| α-Galactosidase | 10,000 | GAL/g |
| β-glucanase | 3500 | BGU/g |
| Cellulase | 80,000 | CU/g |
| Xylanase | 150,000 | XU/g |
| Glucoamylase | 1000 | AG/g |
| Amylase | | |
| Ginger | | |
| Turmeric | | |
| Hemicellulase | 40000 | HCU/g |
| Invertase | 10000 | Sumner/g |
| Pectinase | 3500 | Endo PG/g |
| Peptidase | 500 | LAP/g |
| Phytase | 3000 | U/g |
| Lactococcus lactis and | | |
| Lactococcus cremoris | | |

Pasta was cooked as instructed on the package labels and the recommended portion size was tested. The whole wheat bread was tested at four slices of bread per one liter of water. The foods were mixed with water using a blender and the pH adjusted to 4.0. Samples were transferred to control or experimental flasks. Two capsules equaling a total of 750 mg of the composition described in Table 1 were added to all experimental samples. No composition or additional material was added to control. Both experimental flasks and controls were incubated at 37° C. at 100 RPM shaking for 60 minutes. Samples were taken at 0 minutes, 2 minutes, 30 minutes, and 60 minutes. Gluten was then extracted from the samples and analyzed by an AOAC certified test.

Approximately, 96% of gluten in wheat bread was hydrolyzed within the first few seconds of the addition of the composition according to the disclosure. The amount of wheat bread tested was double the recommended serving size. At 30 minutes the amount of gluten was below 20 ppm and at 60 minutes the concentration of gluten was at 4 ppm.

The hydrolysis of gluten in pasta was more rapid such that than 98% of gluten was broken down in the initial addition of the composition according to the disclosure. At time point 0 the gluten concentration was 14 ppm and then decreased to about 3-4 ppm by 60 minutes.

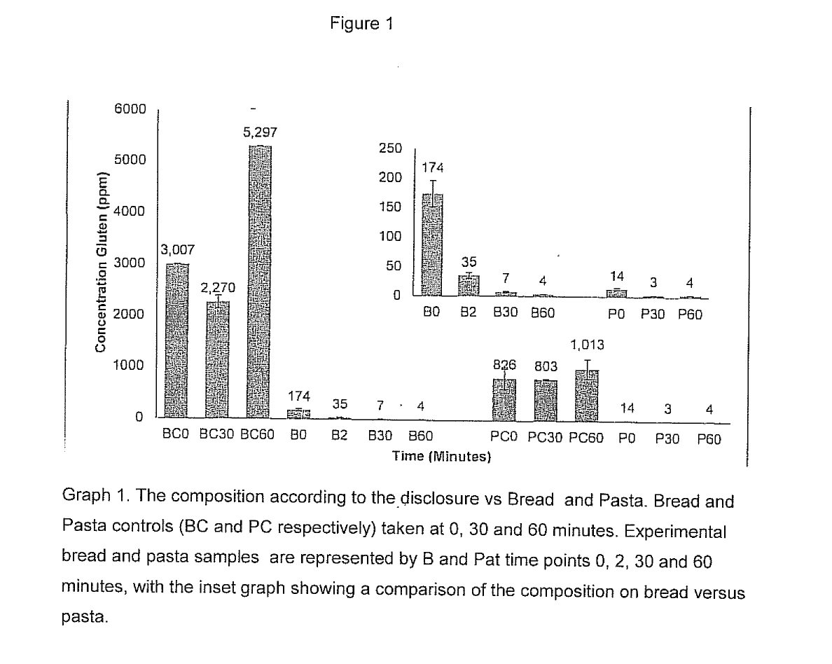

The results are summarized in Table 2 and FIG. 1. In FIG. 1, the results are presented in the form of a bar graph that compares the control samples with the experimental samples and also compares the experimental samples, bread and pasta.

TABLE 2
Summary of results of treatment of pasta and bread with a composition
according to the disclosure. Samples were taken at the indicated
timepoints and assayed for gluten concentration (shown in ppm)

| Sample | | | | |
|---|---|---|---|---|
| (Gluten ppm) | 0 minutes | 2 minutes | 30 minutes | 60 minutes |
| Bread Control | 3007 ± 3 | NA | 2270 ± 125 | 5297 ± 5 |
| Bread plus | 174 ± 22 | 35 ± 5 | 8 ± 1 | 4 ± 0.1 |
| Composition | | | | |
| Pasta Control | 826 ± 215 | NA | 803 ± 13 | 1013 ± 189 |
| Pasta plus | 14 ± 2 | NA | 3 ± 0.2 | 4 ± 0.1 |
| Composition | | | | |

### Example 2

In this example, twenty-seven (27) patients, all of whom met the criteria outlined below in Table 3, were selected to take two (2) capsules of the composition according to the disclosure before two (2) major meals of the day for 2 weeks. Twenty-three (23) patients were female and four (4) were male, with ages ranging from 25-77. The following symptoms were assessed at baseline, week 1, and week 2, the conclusion of the study:
Abdominal pain
Diarrhea
Constipation
Headaches
Joint pain
Fatigue
The severity of symptoms was measured as mild, moderate, or severe, and none if symptoms were absent.

All patients were contacted by phone within 48 hours of start of the trial to assess for any adverse effects. Following parameters were checked at baseline, week 1, and week 2:
Weight
Height
Blood pressure
Pulse rate
Respiration rate

TABLE 3

| Inclusion criteria | Exclusion Criteria |
|---|---|
| Ages 18-80 years | Active Inflammatory Disease |
| Physician diagnosed gluten | Celiac disease confirmed by antibodies |
| sensitivity by history and | and duodenal biopsy• |
| experienced symptoms of | |
| gluten sensitivity for at least | |
| 1 month prior to involvement | |
| Willing to take supplement twice | Peptic ulcer disease |
| daily for 2 weeks | |
| | Lactose intolerance |
| | Pregnant or lactating women |
| | Received any experimental |
| | drug within 30 days of |
| | enrollment |

### Results

The results are presented in tables 4-6. Table 4 presents a summary of the symptoms experienced by the patients at baseline. Table 5 presents a summary after the patients took the composition for one week and Table 6 after two weeks.

Nineteen patients (83%) claimed a marked improvement after taking the composition and four rated an improvement in symptoms after taking the composition. The improvements were seen in abdominal pain, bloating, changes in bowel habits and fatigue. There was no improvement reported in headaches or joint pain. There were no adverse effects reported.

TABLE 4
Baseline (27 patients)

| Symptoms | | | | |
|---|---|---|---|---|
| (number of | | | | |
| patients) | None | Mild | Moderate | Severe |
| Abdominal | 1 | 1 | 16 | 9 |
| Pain/Cramping | | | | |
| Bloating/Distention | 0 | 3 | 9 | 15 |
| Diarrhea | 10 | 4 | 2 | 11 |
| Constipation | 16 | 2 | 3 | 6 |
| Headaches | 11 | 5 | 7 | 4 |
| Joint Pains | 12 | 2 | 9 | 4 |
| Fatigue | 3 | 4 | 5 | 15 |

TABLE 5
Twenty-three patients had the following symptoms at week 1:

| Symptoms | | | | |
|---|---|---|---|---|
| (number of | | | | |
| patients) | None | Mild | Moderate | Severe |
| Abdominal | 10 | 7 | 4 | 2 |
| Pain/Cramping | | | | |
| Bloating/Distention | 9 | 10 | 1 | 3 |
| Diarrhea | 16 | 5 | 2 | 0 |
| Constipation | 20 | 1 | 1 | 1 |
| Headaches | 17 | 2 | 3 | 1 |
| Joint Pains | 14 | 2 | 4 | 3 |
| Fatigue | 7 | 8 | 3 | 5 |

TABLE 6
Twenty-three patients had the following symptoms at week 2:

| Symptoms | | | | |
|---|---|---|---|---|
| (number of | | | | |
| patients) | None | Mild | Moderate | Severe |
| Abdominal | 15 | 4 | 2 | 2 |
| Pain/Cramping | | | | |
| Bloating/Distention | 14 | 6 | 1 | 2 |
| Diarrhea | 21 | 1 | 1 | 0 |
| Constipation | 21 | 1 | 0 | 1 |
| Headaches | 16 | 5 | 1 | 1 |
| Joint Pains | 17 | 3 | 2 | 1 |
| Fatigue | 10 | 7 | 1 | 5 |

### Example 3

In this example, fifty-seven patients, all of whom met the criteria outlined in Table 3 from Example 2, were selected to take two (2) capsules of same composition as Example 2 before two (2) major meals of the day for 2 weeks. In table 7, the results are summarized where following symptoms were assessed at baseline, week 1, and week 2, the conclusion of the study:

TABLE 7

| Age | | | |
|---|---|---|---|
| of | | | Bowel |
| patient | Pain/Cramps | Bloating | Function |
| 62 | Improved | Improved | n/a |
| 32 | Improved | n/a | n/a |
| 28 | n/a | n/a | Improved |
| 60 | Not | Improved | n/a |
| | Improved | | |
| 25 | not | | |
| | available . . . | | |
| 33 | Improved | Improved | n/a |
| 57 | | | |
| 53 | Improved | Improved | Improved |
| 47 | Improved | | |
| 43 | n/a | Improved | n/a |
| 50 | Improved | Improved | n/a |
| 54 | Improved | n/a | n/a |
| 53 | Improved | Improved | Improved |
| 64 | Improved | Improved | Improved |
| 67 | Improved | n/a | Not |
| | | | Improved |
| 50 | | | |
| 56 | Improved | Improved | n/a |
| 45 | Improved | n/a | Improved |
| 47 | Improved | Improved | n/a |
| 45 | Not | Not Imp. | Not |
| | Improved | | Improved |
| 59 | Improved | Improved | Improved |
| 71 | Not | Improved | n/a |
| | Improved | | |
| 59 | Improved | Improved | Improved |
| 32 | Not | Not Imp. | Improved |
| | Improved | | |
| 47 | n/a | Improved | Improved |
| 58 | Improved | Improved | Improved |
| 53 | n/a | Improved | n/a |
| 72 | stopped . . . | | |
| 55 | | | |
| 51 | Improved | Improved | Improved |
| 41 | Improved | Improved | |
| 72 | Improved | Improved | Improved |
| 30 | n/a | Improved | Improved |
| 58 | n/a | n/a | Improved |
| 39 | Not | Improved | n/a |
| | Improved | | |
| 37 | did not start . . . | | |
| 64 | n/a | Improved | Improved |
| 74 | n/a | Improved | Improved |
| 36 | Improved | n/a | n/a |
| 66 | Improved | Improved | Improved |
| 58 | did not start . . . | | |
| 38 | | | |
| 71 | n/a | Improved | n/a |
| 65 | improved | improved | Improved |
| 43 | improved | improved | n/a |
| 42 | Improved | Improved | Improved |
| 28 | | | |
| 35 | Improved | Improved | Improved |
| 40 | | | |
| 53 | | | Not |
| | | | Improved |
| 74 | improved | improved | Improved |
| 35 | | | |
| 47 | Improved | Not Imp. | |
| 61 | | | |
| 74 | | | |
| 58 | | | |

With regard to the foregoing description, it is to be understood that changes may be made in detail without departing from the scope of the present disclosure. It is intended that the specification and depicted example to be considered exemplary only, with a true scope and spirit of the invention being indicated by the broad meaning of the claims.

1. A composition for reducing gluten sensitivity, comprising at least three proteases;
at least one carbohydrase;
at least one probiotic; and
wherein said composition relieves symptoms of gluten sensitivity.

2. The composition of claim 1 further comprising ginger and turmeric.

3. The composition of claim 1 wherein said composition relieves the symptoms of non-celiac gluten sensitivity.

4. The composition of claim 1 wherein said at least three proteases are selected from the group consisting of Protease 3.0, Protease 4.5, Protease 6.0, Papain, Bromelain, Protease Aspergillus oryzae, Protease Aspergillus melleus, Proteases I-V, and DPP IV and combinations thereof.

5. The composition of claim 1 wherein said at least one carbohydrase is selected from the group consisting of lactase, α-galactosidase, β-glucanase, xylanase, glucoamylase, amylase, hemicellulase, invertase and pectinase and combinations thereof.

6. The composition of claim 1 wherein said at least one probiotic is selected from the group consisting of Lactococcus lactis and Lactococcus cremoris.

7. The composition of claim 1 wherein said composition prevents the symptoms of non-celiac gluten sensitivity where said symptoms are selected from the group consisting of abdominal pain, cramping, bloating/distention, diarrhea, constipation and fatigue and combinations thereof.

8. The composition of claim 7 wherein said symptoms of non-celiac gluten sensitivity are selected from the group consisting of abdominal pain, bloating, changes in bowel habits and fatigue and combinations thereof.

9. The composition of claim 1 wherein said composition reduces the concentration of gluten to below about 20 ppm.

10. The composition of claim 1 wherein said composition reduces the concentration of gluten to below about 5 ppm.

11. The composition of claim 1 wherein said composition reduces the concentration of gluten to between about 0.1 ppm to about 5.0 ppm.

12. The composition of claim 1 further comprising lipase.

13. The composition of claim 1 further comprising a phytase.

14. A composition for preventing the effects of gluten in a gluten-sensitive individual, wherein said composition comprises:
at least three proteases selected from the group consisting of Protease 3.0, Protease 4.5, Protease 6.0, Papain, Bromelain, Protease Aspergillus oryzae, Protease Aspergillus melleus, Proteases I-V, and DPP IV and combinations thereof;
at least one carbohydrase selected from the group consisting of lactase, α-galactosidase, β-glucanase, xylanase, glucoamylase, amylase, hemicellulase, invertase and pectinase and combinations thereof;
at least one probiotic selected from the group consisting of consisting of Lactococcus lactis and Lactococcus cremoris and combinations thereof;
Ginger;
Turmeric;
a lipase;
phytase; and
wherein said composition reduces the concentration of gluten to less than about 20 ppm.

15. The composition of claim 14 comprising three proteases and three carbohydrases.

16. The composition of claim 14 wherein said composition relieves the symptoms of non-celiac gluten sensitivity are selected from the group consisting of abdominal pain, cramping, bloating/distention, diarrhea, constipation and fatigue.

17. The composition of claim 14 wherein said composition reduces the concentration of gluten to between 0.1 ppm to 5.0 ppm.

18. A method of reducing non-celiac gluten sensitivity, comprising
(a) Formulating a composition, said composition comprising:
at least three proteases selected from the group consisting of consisting of Protease 3.0, Protease 4.5, Protease 6.0, Papain, Bromelain, Protease Aspergillus oryzae, Protease Aspergillus melleus, Proteases l-V, and DPP IV;
a least one carbohydrase selected from the group consisting of lactase, α-galactosidase, β-glucanase, xylanase, glucoamylase, amylase, hemicellulase, invertase and pectinase and combinations thereof;
a least one probiotic selected from the group consisting of Lactococcus lactis and Lactococcus cremoris and combinations thereof;
ginger;
turmeric;
a lipase; and
phytase
(b) ingesting said composition; and
Wherein said composition relieves the symptoms of non-celiac gluten sensitivity.

19. The composition of claim 1 wherein said symptoms of non-celiac gluten sensitivity are selected from the group consisting of abdominal pain, bloating, changes in bowel habits and fatigue and combinations thereof.

20. The composition of claim 1 wherein said composition reduces the concentration of gluten to between about 0.1 ppm to about 5.0 ppm.
